(19) United States
(12) Patent Application Publication
(10) Pub. No.: US 2026/0174647 A1
(43) Pub. Date: Jun. 25, 2026

(54) DEODORANT COMPOSITIONS

(71) Applicant: Conopco, Inc., d/b/a UNILEVER, Hoboken, NJ (US)

(72) Inventors: Mackenzie KOLMAN, Trumbull, CT (US); Joy Rachel McWALTER, Yorkshire (GB); Kathleen Lynn MELO, Trumbull, CT (US)

(21) Appl. No.: 19/421,879

(22) Filed: Dec. 16, 2025

(30) Foreign Application Priority Data
Dec. 20, 2024 (EP) 24222137.2

(51) Int. Cl. A61K 8/36; A61K 8/02; A61K 8/19; A61K 8/37; A61Q 15/00

(52) U.S. Cl. CPC A61K 8/361; A61K 8/0229; A61K 8/19; A61K 8/375; A61Q 15/00; A61K 2800/30; A61K 2800/34; A61K 2800/48

(57) ABSTRACT

A deodorant composition comprising a carrier material and a deodorant active system comprising a zinc dicarboxylate salt of a fatty acid having from 6 to 18 carbon atoms and a magnesium salt.

### FIELD OF INVENTION

The invention is concerned with deodorant compositions, particularly such compositions having an enhanced activity deodorant active system

### BACKGROUND

Deodorant compositions are known in the art. Such compositions may comprise antiperspirant actives that reduce sweat on the skin surface as well as reducing malodour. Others contain non-aluminium deodorant actives that may reduce body odour by countering odour-causing bacteria on the skin surface or by absorbing malodours. The present invention is largely concerned with deodorant compositions of the latter kind.

Deodorant compositions comprising zinc-based actives are known in the art. Examples of publications disclosing such compositions are given below.

Zinc ricinoleate is a particularly well-known zinc-based deodorant. EP 3878431 A1 (Hyteck, 2021) discloses stable deodorant compositions comprising zinc ricinoleate.

WO 18/087147 A1 (Givaudan, 2018) discloses deodorant compositions comprising zinc neodecanoate and fragrance.

WO 18/087148 A1 (Givaudan, 2018) discloses deodorant compositions comprising zinc neodecanoate and aluminium chlorohydrate.

Deodorant compositions comprising magnesium salts are also known in the art.

WO 2020/205814 A1 (P&G, 2020) discloses aqueous deodorant compositions comprising magnesium salts.

WO 2022/150889 A1 (L′Oreal, 2022) discloses emulsion stick deodorant compositions comprising magnesium salts.

### SUMMARY OF INVENTION

The invention is concerned with deodorant compositions of enhanced activity. The deodorant compositions involved comprise a synergistic blend of a zinc dicarboxylate deodorant active and a magnesium salt.

It is an object of the invention to provide deodorant compositions having superior efficacy.

It is a further object of the invention to provide deodorant compositions that have low irritation potential.

It is a further object of the invention to provide deodorant compositions that have good environmental credentials in terms of biodegradability and/or sustainability.

In a first aspect of the invention, there is provided a deodorant composition comprising a carrier material and a deodorant active system comprising a zinc dicarboxylate salt of a fatty acid having from 6 to 18 carbon atoms and a magnesium salt.

In a second aspect of the invention, there is provided a cosmetic method of obtaining a deodorancy benefit comprising the topical application to the surface of the human body of a deodorant composition according to the first aspect of the invention.

### DETAILED DESCRIPTION

Herein, features expressed as “preferred” with regard to a particular aspect of the invention should be understood to be preferred with regard to each aspect of the invention (likewise, features expressed as “more preferred”, “particularly preferred” or “most preferred”). “Preferred” features aid the delivery of one or more of the objects of the invention.

Herein, preferred features of the invention are particularly preferred when used in combination with other preferred features (likewise, features expressed as “more preferred”, “particularly preferred” or “most preferred”).

Herein, “ambient conditions” refer to 20° C. and 1 atmosphere pressure, unless otherwise indicated.

Herein, water solubility is expressed in grams per litre (g/l) as measured at 20° C. and one atmosphere pressure.

Herein, all percentages, ratios and amounts are by weight, unless otherwise indicated.

Herein, amounts and concentrations of ingredients are percentages by weight of the total composition, unless otherwise indicated.

Herein, references to amounts of components such as “carrier oil” or “thickening agent” relate to the total amount of such components present in the composition.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Compositions of the invention are preferably of low free water content. The free water content of compositions of the invention is preferably less than 1%, more preferably less than 0.1% and still more preferably less than 0.01%. Most preferably, compositions of the invention are anhydrous, meaning that they have zero free water.

Herein, “free water” is any water present in the composition other than that present as water of hydration in solid, typically crystalline, materials present within the composition. Such water of hydration is typically strongly bound to the solid with which it is associated and is not free to interact with other components.

Compositions of the invention do not require aluminum or zirconium salts present to deliver their deodorancy effect. Preferably, compositions of the invention are free from aluminium and zirconium salts.

The magnesium salts used in compositions of the invention are preferably of low water-solubility. Preferred magnesium salts have a water-solubility of less than 1 g/l and more preferably less than 0.5 g/l. Without wishing to be bound by theory, it is thought that magnesium salts having low water-solubility are less likely to undergo performance-reducing interactions with other components of the composition.

Particularly preferred magnesium salts are magnesium hydroxide (water solubility less than 0.01 g/l), magnesium oxide (water solubility less than 0.01 g/l) and magnesium carbonate (water solubility 0.39 g/l).

In some embodiments, compositions of the invention may have both magnesium hydroxide and magnesium carbonate present. In such embodiments, the ratio of magnesium hydroxide to magnesium carbonate is preferably from 1:1 to 25:1, more preferably from 2:1 to 20:1 and most preferably from 5:1 to 10:1. Such ratios may enhance the deodorancy performance of the composition.

The magnesium salt is preferably used at a level of from 0.5 to 50%, more preferably from 1 to 30% and most preferably from 5 to 20%.

An essential part of the deodorant active system used in the invention is a zinc dicarboxylate salt of a fatty acid having from 6 to 18 carbon atoms. Herein, references to zinc carboxylates are to zinc dicarboxylates. Such salts are relative hydrophobic in nature, enhancing the ease with which they can be formulated with other hydrophobic components, particularly in compositions having a low content of free water and especially in anhydrous compositions.

In some embodiments, the C6-C18 fatty acids of the zinc dicarboxylate are selected from those having few hydrophilic groups, notably less than two hydroxyl groups per fatty acid residue. Preferred zinc carboxylates are zinc dicarboxylates of a C6 to C18 fatty acid having zero hydroxy groups per fatty acid residue.

Particularly preferred zinc carboxylate salts used are salts of a fatty acid having from 8 to 14 carbon atoms, especially 8 to 12 carbon atoms.

Preferred zinc carboxylate salts used in the present invention are salts of a saturated fatty acid.

Preferred zinc carboxylate salts used in the present invention are salts of a branched chain fatty acid, particularly when having from 8 to 12 carbon atoms and more particularly when saturated.

An especially preferred zinc carboxylate used in the present invention is zinc neodecanoate, which is a salt of zinc and neodecanoic acid having the formula [C₉H₂₀—CO·O]₂Zn.

The zinc dicarboxylate salt is preferably present at from 0.1 to 20%, more preferably at from 0.5 to 10% and most preferably at from 1 to 7% of the total composition.

The ratio of the zinc-based deodorant active to the magnesium salt is preferably from 1:20 to 1:1, more preferably from 1:10 to 1:2 and most preferably from 1:5 to 1:2. Such ratios may enhance the deodorancy performance of the composition.

The carrier material may be any material with which the deodorant active system may be formulated to aid its topically delivery. It may be considered a cosmetically acceptable carrier material. The carrier material may comprise several components.

A typical carrier material is carrier fluid or carrier oil, optionally formulated with a structuring agent to transform it into a solid form.

Solid deodorant sticks and soft solids are preferred formats for compositions of the invention, in particular solid sticks and especially sticks having a hardness of less than 20 mm, preferably less than 15 mm and more preferably less than 10 mm. Such sticks can demonstrate a combination of aesthetically desirable properties, including sensory benefits such as the avoidance of excessive drag on application and sometimes lower visible marks on clothing worn in close proximity to the area of application on the skin.

Herein, the “hardness” of stick and soft solid compositions refers to the depth, in millimetres, that a cone penetrates into a test specimen under fixed conditions, as determined in accordance with the procedures of ASTM Method D217-48, incorporated herein by reference, using a Petrotest PNR10 Penetrometer (or equivalent), equipped with an ASTMD2884 plunger (Petrotest Cat. #18-0081 or equivalent, weight=47.5 g) and a 2.5 g aluminium cone, 20° angle with a base diameter of 10 mm, wherein hardness values are reported as an average of 6 replicate measurements.

A carrier oil is a preferred carrier material or component of the carrier material. A preferred carrier oil is a natural oil, in particular a triglyceride oil. Silicone oils are preferably absent from the carrier material and the composition as a whole, in order to avoid compromising the environmental credentials of the composition.

Herein, the terms “oil” and “liquid oil” may be used interchangeably and signify a water-insoluble organic material that is liquid at 20° C. Any oil having a solubility of less than 0.1 g/100 g at 20° C. is considered to be water-insoluble.

A preferred oil for use in compositions prepared in accordance with the invention is a fragrance oil, sometimes alternatively called a perfume oil. The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is commonly up to 3% advantageously is at least 0.5% and particularly from 0.8% to 2%.

The total amount of liquid oil in the composition is preferably from 10 to 80%, more preferably from 20 to 70%, and most preferably at from 35 to 65% by weight of the total composition.

In certain embodiments, it is preferred to include an oil, other than a fragrance oil, that has a relatively low viscosity, by which is meant less 250 cS (mm²·s⁻¹). Such oils can improve the sensory properties of the composition on application and can lead to other benefits such as emolliency.

Suitable oils can be selected from alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Suitable oils can include one or more triglyceride oils. The triglyceride oils commonly comprise the alkyl residues of aliphatic C₇ to C₂₀ acids, the total number of carbon atoms being selected in conjunction with the extent of olefinic unsaturation and/or branching to enable the triglyceride to be liquid at 20° C. One example is jojoba oil. Particularly preferably, in the triglyceride oil the alkyl residues are linear C₁₈ groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid, petroselenic acid and stearidonic acid.

Suitable oils can include those derived from unsaturated C₁₈ acids, including coriander seed oil, Impatiens balsimina seed oil, Parinarium laurinarium kernel fat oil, Sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil by virtue of its characteristics is sunflower (seed) oil.

Further suitable oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C₁₂₋₁₅ alkyl benzoate, for example Finsolv TN™ or Finsolv Sun™.

A preferred component of compositions of the invention is a thickening agent, sometimes alternatively referred to as a structurant, gelling agent or gellant. Such agents increase the viscosity of or solidify the composition.

The thickening agent may be selected from any of those known in the art, including those as described further hereinbelow.

The thickening agent preferably comprises a wax, meaning that is a hydrophobic material having a melting point above ambient temperature. Preferably, the melting point of the wax is above 50° C. Preferred waxes are natural materials or naturally-derived materials; more preferably, waxes used in the thickening agent are natural materials.

Preferred waxes for use with the present invention include Helianthus Annus (sunflower) seed wax, Oryza Saliva (rice) bran wax, and Euphorbia Cerifera (candelilla) wax, whether used independently or collectively. Each of these structurant is a natural ingredient, enhancing the environmental credentials of compositions of the invention

Independently or in combination with a wax, the thickening agent or structurant may comprise arrowroot powder. Arrowroot powder is a preferred component of compositions of the invention, particularly at a level of from 5 to 35% and especially at a level of from 10 to 30%. Arrowroot is a natural thickening agent and enhances the environmental credentials of compositions of the invention.

The amount of the structurant or thickening agent used in stick compositions of the invention is preferably from 5%, more preferably from 7% and most preferably from 8%. The upper amount is preferably up to 50%, more preferably up to 40% and most preferably up to 30%. The preferred ranges of thickening agent may be selected from any of the lower and upper amounts indicated in this paragraph.

A preferred additional component of compositions of the invention is a vitamin, such as vitamin B (particularly vitamin B3). Vitamins are preferably included at a total level of from 0.1 to 10% and more preferably at from 0.5 to 7.5%.

Other components that may be included in compositions according to the invention including those described in the following paragraphs.

Wash-off agents may be included, often in an amount of up to 10%, to assist in the removal of the formulation from skin or clothing. Such wash-off agents are typically non-ionic surfactants such as esters or ethers containing a C₈ to C₂₂ alkyl moiety and a hydrophilic moiety comprising a polyoxyalkylene group (POE or POP).

Skin feel improvers (e.g. talc or finely divided high molecular weight polyethylene), may be included, typically in an amount from 1 up to 10%.

Skin benefit agents, such as allantoin or lipids, may be included, typically in an amount of up to 5%.

A highly preferred optional component is a preservative system, such as sodium benzoate/citric acid and/or an antioxidant such BHT (butyl hydroxy toluene), each typically in an amount of from 0.01 to 0.5%.

Compositions of the invention may be prepared by any of the methods known in the art.

### Examples

The deodorant stick compositions detailed in Table 1 were prepared by methods known in the art.

TABLE 1

| | Examples | | | |
|---|---|---|---|---|
| | A | 1 | B | C |
| Components | Parts by wt. | | | |
| Shea Butter | 5.50 | 5.50 | 5.00 | 5.00 |
| Capric/Caprylic | 21.60 | 48.66 | 20.55 | 20.55 |
| Triglyceride | | | | |
| Coconut oil | 22.00 | 3.00 | 34.14 | 32.14 |
| Sunflower seed wax | — | — | 7.00 | 7.00 |
| Rice bran wax | — | — | 7.00 | 7.00 |
| Candelilla wax | 3.60 | 3.60 | — | — |
| Jojoba seed ester | 2.20 | 2.20 | 7.00 | 7.00 |
| Arrowroot powder | 12.00 | 21.82 | 15.00 | 15.00 |
| Silica | — | — | 1.00 | 1.00 |
| Magnesium hydroxide | 21.50 | 8.50 | — | — |
| Magnesium carbonate | 1.90 | 1.20 | — | — |
| Zinc dicarboxylate | — | 2.00 | 2.00 | 4.00 |
| Kaolin clay | 8.50 | 2.82 | — | — |
| Minor components | 0.20 | 0.20 | 0.41 | 0.41 |
| Fragrance | 1.00 | 1.00 | 0.90 | 0.90 |
| MMS (48 hr.) | −1.18 | −1.42 | −0.75 | −0.90 |
| MMS (72 hr.) | −1.04 | −1.31 | −0.49 | −0.66 |

The zinc dicarboxylate indicated was a mixture of zinc neodecanoate (80%), isopropyl myristate (19%) and fragrance (1%).

The “minor components” indicated were non-functional from a deodorancy perspective and consisted of minor amounts of triethyl citrate, essential oils, vitamins, and/or preservatives.

The compositions indicated in Table 1 were each independently tested against a “no treatment control”. The compositions were applied at a dosage of 0.31 g (+/−0.05 g) to the axillae of approximately 40 female panellists. A panel of 4 highly trained sensory evaluators assessed the malodour in the axillae of the panellists 48 hours and 72 hours after application. Malodour scores were assigned on the basis of a 1 to 5 line scale. A statistical analysis of the results produced the Mean Malodour Scores (MMS) indicated at the bottom of Table 1. These are expressed as the MMS achieved with each of the compositions tested relative to the control (no treatment).

Each of the compositions tested produced a significant reduction in malodour (p<0.01) compared with the no treatment control. The best performing composition was Example 1. This gave a reduction in malodour of 1.42 after 48 hours and 1.31 after 72 hour compared with the control. This was better than Comparative Example A which had more than twice the content of magnesium salts. It was also better than Comparative Example C, which had twice the content of zinc dicarboxylate. These results suggest a surprising synergy in deodorancy performance for a deodorant active system comprising a zinc dicarboxylate salt of a fatty acid having from 6 to 18 carbon atoms and a magnesium salt.

1. A deodorant composition comprising a carrier material and a deodorant active system comprising a zinc dicarboxylate salt of a fatty acid having from 6 to 18 carbon atoms and a magnesium salt.

2. The deodorant composition according to claim 1, wherein the zinc dicarboxylate salt is zinc neodecanoate.

3. The deodorant composition according to claim 1, wherein the magnesium salt has a water solubility of less than 0.5 g/l.

4. The deodorant composition according to claim 3, wherein the magnesium salt is selected from magnesium hydroxide, magnesium oxide and magnesium carbonate.

5. The deodorant composition according to claim 4, comprising both magnesium hydroxide and magnesium carbonate.

6. The deodorant composition according to claim 1, wherein the weight ratio of zinc dicarboxylate to magnesium salt is from 1:10 to 1:2.

7. The deodorant composition according to claim 1, which has a content of free water that is less than 1% by weight.

8. The deodorant composition according to claim 1, which is free from aluminum and zirconium salts.

9. The deodorant composition according to claim 1, further comprising a thickening agent or structurant.

10. The deodorant composition according to claim 7 in the form of a stick or soft solid.

11. The deodorant composition according to claim 1, further comprising a natural oil.

12. The deodorant composition according claim 11, wherein the natural oil is a triglyceride oil.

13. The deodorant composition according to claim 1 which is free from silicone oil.

14. The deodorant composition according to claim 1, further comprising a fragrance.

15. A method of obtaining a deodorancy benefit comprising applying topically to the surface of the human body a deodorant composition according to claim 1.
